(19) United States
(12) Patent Application Publication
(10) Pub. No.: US 2024/0261551 A1
(43) Pub. Date: Aug. 8, 2024

(54) MICRONEEDLE PATCH APPLICATOR SYSTEMS, DOCKING PLATFORMS, APPLICATORS AND MICRONEEDLE PATCHES AND METHODS OF MOUNTING MICRONEEDLE PATCHES ON APPLICATORS

(71) Applicant: UPRAX MICROSOLUTIONS B.V., Delft (NL)

(72) Inventors: Bernardus Hendrikus VAN OORSCHOT, Delfft (NL); Koen VAN DER MAADEN, Delft (NL)

(21) Appl. No.: 18/562,430

(22) PCT Filed: May 12, 2022
(86) PCT No.: PCT/NL2022/050259

§ 371 Date: Nov. 20, 2023

(30) Foreign Application Priority Data
May 20, 2021 (NL) 2028261

(51) Int. Cl. A61M 37/00

(52) U.S. Cl. CPC A61M 37/0015; A61M 2037/0023; A61M 2037/0061

(57) ABSTRACT

A microneedle patch applicator system comprises an applicator for applying, by a user of the system, a microneedle patch to a skin of a subject. The applicator comprises a coupling interface for coupling to the microneedle patch when the applicator is brought in a predetermined coupling position relative to the microneedle patch. The microneedle patch is initially a pre-application state, in which the microneedle patch is not ready-for-application to a skin of a subject, and the applicator is arranged to transfer the microneedle patch from the pre-application state into a state ready-for-application, such that the microneedle patch is in said ready-for-application state when the microneedle patch is coupled to the applicator and the applicator has been moved out of the coupling position.

### FIELD OF THE INVENTION

This invention relates to microneedle patch applicator systems, docking platforms, applicator and microneedle patches, as well as methods of mounting microneedle patches on applicator and methods of applying microneedle patches.

### BACKGROUND OF THE INVENTION

A common technique for delivering drugs from a subject across a biological barrier is the use of a hypodermic needle, such as those used with standard syringes or catheters, to transport drugs across (through) the skin. While effective for this purpose, hypodermic needles generally cause pain; local damage to the skin at the site of insertion; bleeding, which increases the risk of disease transmission; and a wound sufficiently large to be a site of infection. The withdrawal of bodily fluids or other samples, such as for diagnostic purposes, using a conventional hypodermic needle has these same disadvantages. Hypodermic needle techniques also generally require administration by one trained in its use. The needle technique also is undesirable for long term, controlled continuous drug delivery.

Another delivery technique is the transdermal patch, which usually relies on diffusion of the drug across the skin. However, this method is not useful for many drugs, due to the poor permeability (i.e. effective barrier properties) of the skin. The rate of diffusion depends in part on the size and hydrophilicity of the drug molecules and the concentration gradient across the stratum corneum. Few drugs have the necessary physiochemical properties to be effectively delivered through the skin by passive diffusion. Lontophoresis, electroporation, ultrasound, and heat (so-called active systems) have been used in an attempt to improve the rate of delivery. While providing varying degrees of enhancement, these techniques are not suitable for all types of drugs, failing to provide the desired level of delivery. In some cases, they are also painful and inconvenient or impractical for continuous controlled drug delivery over a period of hours or days. Attempts have been made to design alternative devices for active transfer of drugs, or analyte to be measured, through the skin.

As an alternative transdermal delivery technique, microneedle patches have been developed. Microneedle patches are patches with, very small, structures, typically shorter than 1 mm, which can be pressed onto the skin of a subject and pierce the skin, see e.g. McConville, Aaron et al. “Mini-Review: Assessing the Potential Impact of Microneedle Technologies on Home Healthcare Applications.” Medicines (Basel, Switzerland) vol. 5,2 50. 8 Jun. 2018, incorporated herein by reference. Through the pierced skin, drugs or other substances may then be delivered into the body of the subject, or alternatively samples be taken from the body.

Although various types of applicators for microneedle patches are known, such as from European Patent EP 2 906 285, up to now their performance is unsatisfactory for many applications. In particular, for applicators manufactured without a pre-mounted microneedle patch, it is difficult for the person applying the microneedle patch to the subject to mount the microneedle patch on the applicator without damaging the microneedles or contaminating the sterile parts of microneedle patch.

### SUMMARY OF THE INVENTION

The present invention provides microneedle patch applicator systems, docking platforms, applicators and microneedle patches, as well as methods of mounting microneedle patches on applicator and methods of applying microneedle patches as described in the accompanying claims. Specific embodiments of the invention are described in the dependent claims.

These and other aspects of the invention will be apparent from and elucidated with reference to the embodiments described hereinafter.

### BRIEF DESCRIPTION OF THE DRAWINGS

Further details, aspects and embodiments of the invention will be described, by way of example only, with reference to the drawings. In the drawings, like reference numbers are used to identify like or functionally similar elements. Elements in the figures are illustrated for simplicity and clarity and have not necessarily been drawn to scale.

FIG. 1 schematically shows a, cross-sectional side view of an example of an embodiment of a microneedle patch applicator system.

FIG. 2 schematically shows a cross-sectional side view of an example of a docking station and an example of a microneedle patch prior to docking.

FIG. 3 schematically shows a cross-sectional side view of an example of a docking station with an example of a microneedle patch which has been docked thereon.

FIG. 4 schematically shows a cross-sectional side view of an example of a docking station and an example of a microneedle patch in a pre-application state, with an example of an applicator positioned to transition the microneedle patch to a state ready-for-application.

FIG. 5 schematically shows a cross-sectional side view of the example of FIG. 4, with microneedle patch coupled to the applicator.

FIG. 6 schematically a cross-sectional side view of the example of FIG. 4, with microneedle patch in the state-ready for application.

FIG. 7 schematically shows a cross-sectional side view of an example of an applicator with a microneedle patch in the state-ready for application placed on the skin of a subject.

FIG. 8 schematically shows a cross-sectional side view of an example of a microneedle patch applied to the skin of a subject.

FIG. 9 schematically shows exploded, perspective views of an example of a microneedle patch in a pre-application state.

FIG. 10 schematically shows a cross-sectional side view of an example of a docking platform with a microneedle patch in a pre-application state coupled to an applicator.

FIG. 11 schematically shows a cross-sectional side view of an example of a docking platform during docking of a microneedle patch.

FIG. 12 schematically shows a cross-sectional side view of an example of a docking platform with a microneedle patch docked thereon.

FIG. 13 schematically shows a cross-sectional side view of an example of an embodiment of a docking station.

FIG. 14 schematically shows a perspective view of another example of an embodiment of a docking station.

FIG. 15 schematically shows a perspective view of yet another example of a docking station.

FIG. 16 schematically shows a perspective view of an upper part of the example of FIG. 15 with a microneedle patch mounted thereon.

FIG. 17 schematically shows a partial, perspective side view of an example of a latch of the example of FIG. 15.

FIG. 18 schematically shows a side view of the upper part of the example of a latch of FIG. 17.

FIG. 19 schematically shows a top view of an example of a package for microneedle patches.

FIG. 20 shows a cross-sectional side view of a skin of a subject with different examples of patches provided thereon.

### DETAILED DESCRIPTION OF THE PREFERRED EMBODIMENTS

Because the illustrated embodiments of the present invention may for the most part, be implemented using materials and shapes known to those skilled in the art, details will not be explained in any greater extent than that considered necessary for the understanding and appreciation of the underlying concepts of the present invention and in order not to obfuscate or distract from the teachings of the present invention.

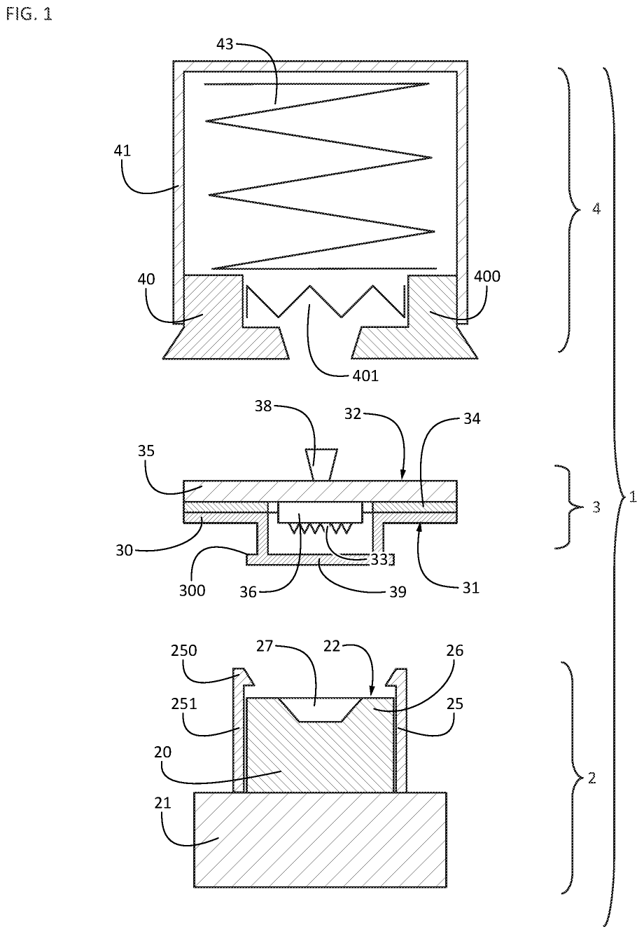

Referring to FIG. 1, the example of a microneedle patch applicator system 1 shown therein comprises a docking platform 20 and an applicator 4, but in an alternative implementation the docking platform 20 may be omitted. In the shown example, the docking platform 20 is part of a docking station 2, but this is not required either. Also shown in FIG. 1 is a microneedle patch 3, and the system 1 may be provided with or without such a patch. The system 1 may e.g. be used for self-application by the subject of the microneedle patch 3, without intervention of a medical practitioner, in which case the user of the system and the subject are the same.

The microneedle patch 3 can be coupled to the applicator 4 while the microneedle patch 3 is in a pre-application state, in which the microneedle patch 3 is not ready for application to a skin 5 of a subject. In the illustrated examples, for instance, in the pre-application state the microneedles 33 cannot penetrate the skin and/or the microneedle patch cannot be attached to the skin, in this example because the microneedles 33 and/or the skin-adhesive surface 34 of the microneedle patch 3 are covered by a removable part 30 of the microneedle patch. The pre-application state can be the original, factory condition of the microneedle patch 3 but alternatively some operations may have taken place, e.g. such as taking the microneedle patch 3 out of a packaging. The patch 3 also has a ready-for application state in which the microneedle patch 3 is ready for application to the skin of a subject. In the shown example, in this state the microneedles 33 and/or the skin-adhesive surface 34 are exposed, allowing the skin to be penetrated and/or to attach the patch to the skin.

Using the system 1, a microneedle patch 3 can be mounted on the applicator 4 and the microneedle patch 3 can be transferred from the pre-application state into the ready-for application state. The system allows to mount microneedle patch on the applicator with little risk of damaging the microneedles or contaminating the microneedles, e.g. without the user touching the microneedle patch 3. In particular, if the system is implemented with a docking platform, docking the microneedle patch 3 is a relatively simple operation, which is performed with microneedle patch not ready for application while with the applicator the microneedle patch 3 is not only taken from the docking platform 20 and mounted, but also brought into the ready-for-application state. The risk of damaging the microneedles or contaminating microneedle patch is therefore limited.

In the shown example, the microneedle patch 3 can be docked on the docking platform 20 while in the pre-application state, and once docked be coupled to the applicator 4. In an alternative embodiment, for instance, the applicator 4 may e.g. pick the patch 3 directly, for example using a snap-fit connection which interlocks with the microneedle patch 3 when the applicator 4 is placed over the patch.

Upon or after being coupled to the applicator 4, the microneedle patch 3 may be transferred into the ready-for-application state. For example, the applicator 4 may comprise a manipulator which manipulates the microneedle patch 3 to be ready for application or, as in this example, the applicator 4 and docking platform 2 may collaborate to transfer the patch.

The transfer may be a one-way transfer, as in the shown examples, but in an alternative example the system 1 may be implemented to bring the microneedle patch 3 from the ready-for application state into a not-ready-for-application state as well, e.g. the pre-application state or another not-ready-for-application state. For example, one or more of the applicator, docking station, or a separate tool may be implemented to further take the microneedle patch 3 from the skin after application, e.g. when substances from the subject have been collected, and the docking platform 20 be implemented to hold a cover which attaches to the removed patch 3 when the applicator 4 positions the patch 3 on the cover, to allow the microneedle patch 3 to be brought back from the ready-for application state into a sealed state in which at least the microneedles of the patch 3 are covered to avoid contamination of the samples collected, and/or to protect anyone from contamination with the substances on the samples when handling the patch.

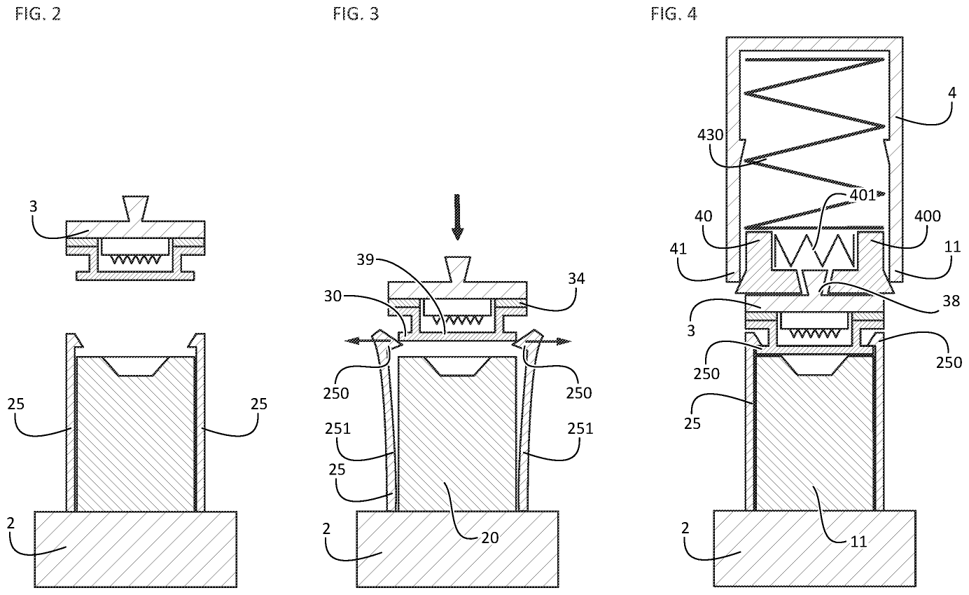

To mount the microneedle patch 3 on the applicator, the applicator 4 comprises a coupling interface 40 for coupling to the microneedle patch 3. As explained in more detail with reference to FIGS. 2-6, the coupling interface 40 automatically couples the microneedle patch 3 to the applicator 4, when the applicator 4 is in a coupling position relative to the patch 3. In the shown example, the applicator couples to the microneedle patch 3 when all of the following conditions (i)-(ii) are met:
(i) the microneedle patch 3 is docked on the docking platform 20, such as after performing the operations illustrated in FIGS. 2 and 3, and
(ii) the applicator 4 is brought in a predetermined position relative to the docking platform 20, hereinafter also referred to as the coupling position, such as by performing the operations illustrated in FIGS. 4 and 5.

It will be apparent that once coupled, the microneedle patch 3 is to remain coupled to the applicator until applied to the skin and is to be separated from the applicator while remaining applied to the skin. In this example, the microneedle patch 3 remains coupled to the coupling interface until application to the skin. In alternative implementations the microneedle patch 3 may, while remaining retained to the applicator, be uncoupled and coupled to other parts inside the applicator, e.g. as part of a series of manipulations that bring the microneedle patch into a state ready-for-application, before application to the skin.

The applicator is arranged to transfer the microneedle patch from the pre-application state into a state ready-for-application, such that the microneedle patch is in the ready-for-application state when the microneedle patch is coupled to the applicator and the applicator has been moved out of the coupling position. This allows to avoid touching or other manual acts by the user of the system that risk to damage the microneedles or contaminate the microneedles for instance. The applicator may then be used to apply the microneedle patch 3 to the skin 5 of a subject.

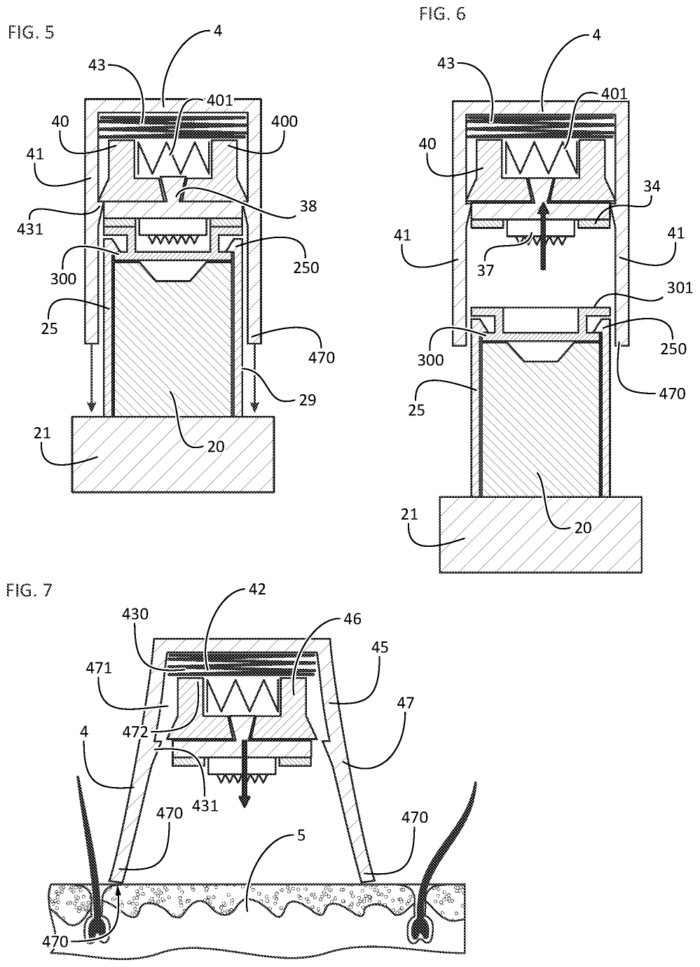

It will be apparent though that alternatively the applicator may by itself bring the patch 3 into the state ready-for-application. For example, the applicator may comprise a manipulator which holds cover 39 in position relative to the sheath 41, e.g. by an interlock with a snap-fit connector of the applicator 4, while the remainder of the patch is moved further inwards into the sheath, for instance. In the shown example though, the docking platform 20 and the applicator 4 are arranged to collaborate to automatically transfer the microneedle patch 3 from the pre-application state into the ready-for-application state. The microneedle patch 3 will in this example in the ready-for-application state when all of the following conditions (iii)-(iv) are met:
(iii) the microneedle patch 3 is coupled to the applicator 4, e.g. by performing the operations illustrated in FIG. 5; and
(iv) the applicator 4 has been moved out of the, predetermined, coupling position (and the microneedle patch 3 has been taken from the docking platform 20), e.g. by performing the operations illustrated in FIG. 6.

For instance, the docking platform 20 and the applicator 4 may be arranged to transfer the microneedle patch 3 into the ready-for application state without any human contact to the microneedle patch 3 at all. The system may allow to separate the removable part 30 and expose the microneedle(s) 33 without the user touching the microneedle patch 3. The risk of contamination of the microneedle patch 3 prior to application of the microneedle patch 3 to the skin 5 of the subject can therefore be reduced.

To ensure a proper positioning and/or orientation of the applicator 4 in the coupling position, the system 1 may comprise a guide 29 for the applicator 4. The guide 29 defines a predetermined path for moving the applicator 4 from an initial position in which the applicator 4 engages with the docking platform 20 into the coupling position. The applicator 4 may be freely movable relative to the docking platform 20 when not engaged with the guide 29. In the example for instance, the applicator 4 comprises a sheath 41 which can slide over the platform 20. The outer shape of the platform 20 thus defines the path along which the applicator 4 moves. In this example the outer shape is a straight cylinder, which can for example have an outer diameter 90% or more, but less than 100%, of the inner diameter of the sheath 41. The guide 29 in this example thus defines a straight path which extends from the top 22 downwards to the point whether the platform 20 is maximally admitted into the sheath 41, which is the point illustrated in FIG. 5. As illustrated in FIGS. 4 and 5, the applicator 4 engages with the guide 29 when the skin-side end 470 slides over the top 22 of the platform 20. The sheath 41 is then guided to move towards the base 21 until the coupling position is reached. Depending on the specific implementation, the coupling position can for example be: the position in which the skin-side end 470 touches the base 21; or the position in which the interface 40 is moved to a predetermined depth into the housing; or the position in which the interface 40 is close enough to couple to the interface of the patch 3 docked on the docking platform 20.

In this respect, the docking platform 20 and the applicator 4 may be movable relative to each other to bring the applicator 4 into and out of the coupling position by manual force of the user only. In such a case, there is no need to e.g. provide batteries or other power sources to drive a motor or other non-human powered actuation. This in turn allows for a system 1 which is always available to the user. The docking platform 20 and the applicator 4 can for example be arranged to transfer the microneedle patch 3 from the pre-application state into the ready-for application when a predetermined manual manipulation of the docking platform 20 and/or the applicator 4 is performed by the user. E.g. as in the examples, this transfer may take place during moving the applicator 4 manually out of the coupling position. Alternatively, this transfer may take place when the applicator is manually placed into the coupling position. In the latter case, for instance, a cover may be automatically cut that covers a part of the patch, e.g. a skin-adhesive surface, to leave the part exposed.

The docking platform 20 may be implemented in any manner suitable to dock the microneedle patch 3 and to cooperate with the applicator 4 to transition microneedle patch from the pre-application state to the ready-for application state. As illustrated in FIG. 1, the system 1 may e.g. comprise a docking station 2. Shown in FIG. 1 is a docking station 2 which comprises a base 21 on top of which the docking platform 20 is located.

In the shown example, the docking platform 20 has a top side 22 on which the microneedle patch 3 can be positioned. To that end the top side 22 is in this example shaped as a table 26 on which the microneedle patch 3 can be placed. As explained below in more detail, the table 26 supports a removable part 30 of the patch 3. The table 26 may comprise a depression 27 for admitting the removable part 30. The depression may have a shape complementary to the microneedle side 3 of the patch. The table 26 is in this example provided with a bowl-shaped depression 27 located at the position corresponding to that of the microneedles of the patch 3. When the patch is docked, the microneedles are thus suspended above the depression 27. Thereby, the microneedles have some limited freedom of movement which allows to avoid damage to the microneedles during operation of the system 1. In this example, the microneedles are shielded and separated from the depression by the cover, and the depression allows the cover to move into the depression instead of being pushed against the microneedles. In an implementation, the depression 27 may be accessible by fingers of the user to remove (parts of) the patch 3. E.g. cover 39 may be removed from the docking platform after the cover has been separated from the patch. by popping the cover from beneath out of the latch 25, for example.

FIG. 1 further shows that the docking platform 20 may comprise a latch 25 arranged to latch the microneedle patch 3 onto the docking platform 20 when placing the patch 3 thereon. In an alternative, the latch 25 may unlatch when the applicator takes the mounted patch 3, e.g. when the applicator enters the coupling position or is moved out of the position. However, in the shown example, the latch 25 does not release upon taking the applicator 4 out of the coupling position. This causes a separation of the patch 3 in a part, hereinafter referred to as the removable part 30, retained by the latch, and the other parts of the patch which are coupled to the interface 40 and taken with the applicator 4. The latch 25 can e.g. latch the removable part 30 and not the other parts of the microneedle patch 3. Thus, when a force may be exerted on the other parts to move them away from the docking platform 20, the latch 25 will not hold these parts on the docking platform but only the removable part 30. The bonding between the removable part 30 and the other parts of the patch may be relatively weak, in which case the separation will be along a pre-defined boundary. In the shown example for instance, the bonding between the cover 39 and the other parts is relatively weak, causing the cover to separate from the other parts and to be retained on the platform 20.

In this example, the removable part 30 has to be removed to bring the microneedle patch 3 into the ready-for-application state. As shown, the removable part 30 may for example inhibit the proper use of the microneedles 33, and in this example covers them. The covered microneedles 33 cannot penetrate the skin, and the microneedle patch 3 is therefore not ready for application. Additionally, or alternatively, the removable part 30 may e.g. render the microneedle patch 3 non-adhesive such that the microneedle patch cannot be tacked to the skin. The latch 25 retains the removable part 30 on the docking platform 20 when the applicator 4 is moved out of the coupling position, and the microneedle patch 3 is taken from the docking platform 20. The patch 3 is thus brought into the ready-for-application state.

Shown in FIG. 1 is a microneedle patch 3 which is in a pre-application state. The shown example of a microneedle patch 3 has a microneedle side 31 and an applicator coupling side 32 opposite to the microneedle side 31. In this example, the microneedle side 31 faces the docking platform 20 when docked, and the applicator coupling side 32 faces away from the base 21 and the docking platform.

In this example, the patch 3 comprises a backing 35 located at the applicator coupling side 32 of the patch. The backing 35 is provided with a coupling interface 38, formed in this example by a knob. At the microneedle side 31, the patch 3 is provided with one or more microneedles 33. In this example, the backing 35 is provided at the microneedle side 31 with a microneedle plate 36 from which the microneedles project. Outside the region covered by the microneedle plate 36, the backing 35 has a skin-adhesive surface 34 which completely or partially is covered with a biocompatible adhesive, more specifically a dermatologically acceptable skin-adhesive.

At the microneedle side 31 the removable part 30 is present, which can be separated from the backing 35 in this example. The removable part 30 comprises a cover 39 which in the pre-application state covers at least a part of the microneedle side 31, and in this example covers the skin-adhesive surface 34 and the microneedles 33. The cover 39 forms a protective spacing in which the microneedles 33 are present, as shown the side of the cover 39 facing the microneedles side 31 forms together with the backing 35 a sealed-off chamber in which the needles 33 are admitted.

As shown, the outside of the cover 39 can be shaped to engage with the latch 25. In this example, the cover is profiled, and more specifically provided with a projecting rim 300 which in the direction from the application coupling side 32 to the microneedles side 31 is at a distance from the base of the cover, and which projects from the cover in a projecting direction perpendicular to this direction.

Although other shapes are possible, as can more clearly be seen in FIG. 9, the cover 39 has a plate or saucer-like shape. The lip of the plate-shape covers the adhesive surface, whereas the well of the plate-shape covers the microneedles 33 and forms the cover-side of the seal-off chamber. The underside of the plate-shape, which when docked faces the docking platform 20 and in this example lies on the table 26, is provided with the rim 300. The rim 300 is an annulus which encloses the well of the plate-shape and projects from the underside in a direction parallel to the lip of the plate.

FIG. 1 further shows an applicator 4 which comprises a housing, formed by a sheath 41. The applicator 4 has a a coupling interface 40 for coupling to the microneedle patch 3. In the example of FIG. 1, the microneedle patch 3 comprises a coupling interface 38 compatible with the coupling interface 40 of the applicator 4. Although any interface suitable to physically couple the patch to the applicator may be used, such as adhesive, magnetic or electrostatic coupling interfaces, in the shown example the interfaces 38,40 are mechanical.

As shown, the interface 40 comprises coupling blocks 400. The coupling blocks 400 couple to the interface 38 when the applicator 4 is in the predetermined position. In this example, the blocks 400 are prior to coupling, spaced apart and even when the interface 40 is placed against the interface 38 do not establish the coupling. However, by moving the applicator 4 into the predetermined position, the coupling blocks 400 are brought closer to each other such that the blocks 40 will clamp a protruding part of the interfaced 38 between them. In this example, for instance the interface 38 comprises a knob which can be positioned in the spacing between the blocks 400, and the blocks 400 are moved towards each other to grab the knob, thereby creating an interlocking between the interfaces 38,40.

In the shown example, prior to coupling, the blocks 400 may be kept spaced apart by a resiliently or plastically deformable element. In FIG. 1 for instance a separating spring 401 is schematically indicated which keeps the blocks 400 separated. Upon bringing the applicator 4 into the predetermined position the deformable element deforms under the forces exerted by the blocks 400 thereon, allowing the blocks 400 to move towards each other.

In FIG. 1, the interface 40 is movable relative to the housing of applicator 4, formed by the sheath 41 in this example. The interface is brought into interlock by a movement of the interface 40 relative to the housing. As shown, the blocks 400 have inclined sides, which prior to bringing the applicator 4 in the predetermined position project out of the housing, and which are positioned to contact a perimeter of an opening into the housing in which the interface can be admitted. When the interface 40 is pushed into the housing, the inclined sides contact the perimeter and slide into the housing, causing the blocks 400 to move in a direction perpendicular to the direction of the inwards movement of the interface, bringing the blocks closer together.

In the shown example, the interface 40 has an initial position and is moved into the interlocking state by bringing the applicator 4 into the predetermined position, as illustrated in FIG. 5. The interface 40 is then pushed into the housing by a force exerted by the docking platform 20 opposite to the force required to bring the applicator 4 into the predetermined position relative to the housing. When the patch 3 is on the docking platform 20, as illustrated in FIG. 4, the top side 32 of the patch 3 pushes onto the interface 40, whereas the housing 41 is moved in the opposite direction, causing the interface 40 to move relative to the housing. In this example, the housing 41 is slidable over the top-side 22 of the docking platform 20.

Further indicated in FIG. 1 is an energy storage 43, represented by a spring, inside the housing. The energy storage 43 serves to store energy to drive an actuator 42. This reduces the amount of force that the user has to exert on the skin of the subject, and the associated discomfort for the subject. As illustrated in FIG. 7, the applicator 4 may comprise an actuator 42 for actuating, when in operation, a movement of the microneedle patch 3 in the ready-for-application state relative to the skin 5 to penetrate at least into, or through, the stratum corneum of the epidermis of the skin 5 with the microneedle(s) 33.

Although alternatively, e.g. a battery or other pre-charged power-source may be used depending on the specific type of actuator, in the present example the storage 43 is initially empty and the energy is stored therein by bringing the applicator 4 in the predetermined position. To that end, the applicator 4 may comprise a transducer 44 for converting kinetic energy of the applicator 4 into potential energy stored in the energy storage 43. As illustrated in FIGS. 4 and 5, for example, a biasing spring 430 may be present which is coupled to the coupling interface 40 and biased by a movement of the coupling interface towards the housing, thus converting and storing the kinetic energy in elastic potential energy. In the shown example, the movement brings the interface inside the housing, and the spring exerts a biasing force in the direction opposite to the direction of movement. As more clearly seen in FIG. 5-7, the movement of the coupling interface 40 in the direction of the biasing force is latched, thus holding the spring biased. As shown in FIG. 7, the latching is released when positioning of the applicator 4 on the skin, causing the spring to exert the biasing force on the coupling interface 40, and on the patch 3 causing the patch 3 to move relative to the housing towards the skin 5 and to apply the microneedle patch 3 to the skin 5. The spring is thus both the transducer, energy storage and the actuator. This provides a simple construction.

Referring now to FIGS. 4 and 5, the applicator 4 may comprise a sheath 41 for sliding over the top-side of the docking platform 20. As illustrated in FIG. 3, the latch 25 may comprise a resiliently or plastically deformable fastener which at least during some part of docking the patch 3 projects outwards, in a direction perpendicular to a sliding direction of the sheath 41 and allows the patch to pass and then moves inwards to latch the patch. The sheath 41 when slid over the top-side blocks an outwards movement opposite to the inwards movement and thereby locks the latch 25. In FIGS. 1-7, for example the fastener comprises a flexing lever 251 with an inwards, hooking projection 250. Upon docking the patch, the lever flexes outwards and once the patch is on the platform flexes back to hook behind the rim 300 and latches the patch. whereas in other examples the fastener may e.g. be a part of the platform 20 itself shaped as a snap-fit connector.

In the shown examples the fastener 28 is not moved by the sheath but alternatively, the sheath fastener 28 may be arranged to cooperate with the sheath 41 to be moved inwards upon sliding the sheath 41 over the top-side and thereby latch the removable part 30 on the docking platform 20. For instance, the bend shape illustrated in FIG. 3 may be the natural, not deformed state of the levers (and not as in FIG. 3 a flexed, temporary position caused by the patch 3 pushing the lever outwards). In such a case, the sheath may slide over the lever to flex it inwards and at the same time both latch the patch on the platform and lock the latch.

Referring to FIGS. 6 and 7, indicated therein is that the applicator 4 may comprise a housing 45, and a movable platform 46 for holding the microneedle patch 3 when coupled to the applicator 4. As mentioned above, in this example the sheath 41 forms a housing but the housing may be implemented in any other suitable manner. The movable platform 46 is formed in this example by the bottom side of the interface 40 when coupled to the interface 38, and more specific by the blocks 300 being moved towards each other, grabbing the knob and forming a platform for the microneedle patch. As explained above, the platform is moved relative to the housing 45 when the applicator 4 is brought into the predetermined position in the direction opposite to the direction of the movement of the applicator 4 relative to the docking platform 20.

The movement of the movable platform 46 is used to store energy in the applicator which upon application on the skin is used to actuate movement of the movable platform 46 towards the skin. To that end, in the shown example the energy storage 43 comprises a bias spring 430 which is resiliently deformed by the movement of the movable platform 46, storing the potential energy upon bringing the applicator 4 into the predetermined position. The shown example has a spring latch 431 for latching the spring in deformed state when the microneedle patch 3 is transferred to the state ready-for-application and for releasing the spring to relax and actuate the movement of the movable platform 46 upon a point in time selected by the user, to place the microneedle onto the skin 5 and penetrate the skin 5 with the microneedle(s) 33.

As illustrated in FIG. 5-7, in this example the latch 431 is formed by respective protrusions which block the movement of the movable platform 46 towards the skin-side end 470 of the applicator 4 once the movable platform 46 has been moved into the housing 41 beyond the protrusions. In this example the platform 46 is wider than the distance between the protrusions. To allow the platform to pass, for instance, the blocks 400 may be pushed closer towards each other, in this example by the sliding of the inclined surfaces over the protrusions. Alternatively, for instance, the housing with the protrusions may be elastically deformable to allow the platform 46 to pass.

As indicated in FIG. 7, the applicator 4 may comprise a base 47 for positioning onto the skin 5. In the shown example, the housing 45 is part of the base 47. The base 47 may comprise a skin-side end 470 and a holder 471 for holding the microneedle patch 3 in the ready-for application state in position relative to the skin-side end 470. In the shown example, the holder 471 is formed by a part of the housing 45 at a distance from the skin-side end, in which the movable platform 46 is retained after the applicator 4 is moved out of the predetermined position. The base 47 may be further provided with an interface 472 for an actuator 42, for actuating a movement of the microneedle patch 3 relative to the skin-side end 470 to penetrate at least into the stratum corneum of the epidermis of the skin 5 with the microneedle(s) 33. As shown, in this example upon positioning the applicator 4 on the skin 5, the spring latch 431 is released. As a consequence, the bias spring 430 can push the movable platform 46 from the holder 471 to the skin-side end 470 of the applicator, causing the microneedles 33 to penetrate the skin 5.

The example of FIG. 1 or another system 1 may be used in a method of mounting a microneedle patch 3 on an applicator 4. In case the system is provided in a package, the docking station and/or patch and/or applicator may be taken out of the package before. The method may e.g. be performed by the subject to auto-apply the microneedle patch 3 to a selected body part. The microneedle patch may be provided separately, e.g. in a package containing several patches, and e.g. be taken out of the packaged or docked immediately from the package, possibly by placing the package on a more structural sound part, e.g. a table top or other support, to provide structural strength to the package.

Referring to FIGS. 2-3, such a method may comprise a docking platform 20 holding the microneedle patch 3 in the pre-application state. In the example, for instance, the microneedle patch 3 is docked on the docking station 2 by positioning a microneedle patch 3 on the docking platform 20. In FIG. 2, the microneedle patch 3 is at a distance from the docking platform 20. In FIG. 3, the microneedle patch 3 is being placed on the top-side 22 of the docking platform 20. Although the docking platform may be implemented in various other manners, in the shown example the docking platform 20 has a table shaped top 22 on which the docket patch 3 can lay. The table 26 has a depression 27 which allows the part of the microneedle patch 3 extending over the depression to bent downwards, into the depression. As illustrated in FIG. 2, when docking the microneedle patch 3, a latch 25 will latch the microneedle patch 3 on the docking platform 20, in this example onto the top-side 22 thereof. Although the latch 25 may be implemented in any other suitable manner, in this example the latch 25 comprises levers 251 which can flex in a direction away from the direction of movement of the microneedle patch 3 to allow the microneedle patch to pass. As shown, the levers 251 have a free-end with projections 250 that project inwards, towards the topside and (in this example) the table 26. The microneedle patch pushes against the levers, causing them to flex and the projections 250 to move away. As shown, the projections 250 move back when the levers flex back, causing the projections to hook into the microneedle patch and latch this in position.

FIG. 4 shows the microneedle patch 3 latched in position on the docking platform. As illustrated in FIGS. 4 and 5, the applicator may be brought in the predetermined position relative to the docking platform 20, thereby coupling the coupling interface 40 of the applicator 4 to the microneedle patch 3. In FIG. 4, the applicator is not yet in the predetermined, coupling position, whereas in FIG. 5 the applicator is in the coupling position. As shown, the predetermined position is one in which the coupling interface 40 is at the same height as the coupling interface 38 of the microneedle patch 3, and the applicator 4 projects downwards beyond the topside of the docking platform 2. In this example, as can be seen in FIG. 4, the coupling interface 40 is moved inwards into the applicator 4 when the applicator is moved downwards to project beyond the top-side 22. As can be seen in FIGS. 4 and 5, the inward movement causes the interfaces 38, 40 to couple. More specifically, the top side 22, with the microneedle patch 3 thereon, prevents the coupling interface from moving downwards. Thus when the bottom side of applicator 4 is moved further downwards, the interface 40 moves in the opposite direction relative to the bottom side 4. The applicator 4 transforms this movement in a locking of the interface 40 to the interface 38.

In the shown example, the coupling is a mechanical coupling, caused by parts of the applicator moving to establish an interlocking with the coupling 38 of the microneedle patch 3. The coupling interface 40 may automatically couple to the microneedle patch 3 in another manner when conditions (i) and (ii) are met. For example, the applicator 4 may have an adhesive, magnetic or electrostatic surface which contacts a surface of the microneedle patch, and which exerts a coupling force on the microneedle patch once the applicator is in the predetermined position. The coupling force is preferably smaller than the adhesive force between the microneedle patch and the skin. In addition, the coupling force is preferably stronger than the force that holds the the microneedle patch 3 to the dock. In the shown example for instance, the coupling force is preferably larger than the binding between the removable part and the other parts of the microneedle patch 3.

As illustrated in FIGS. 5 and 6, the microneedle patch 3 may be transferred from the pre-application state into a ready-for-application state upon moving the applicator 4 out of the predetermined position. Alternatively, the transition may be caused by bringing the applicator 4 into the predetermined position. In the shown example, for instance, moving the applicator 4 out of the predetermined position causes a separating force to be exerted on the microneedle patch. On the one hand, the latch 25 engages on a part of the microneedle patch 3, whereas the coupling interface 40 acts on the coupling interface 38 of the microneedle patch. Thus, when the separating force is large enough and exceeds the binding force, these parts will separate. The part on which the latch 25 engages is the removable part 30, and accordingly this is separated from the microneedle patch 3, while the microneedle patch 3 remains mounted in the applicator 4 but now with the microneedles 33 exposed and hence ready for application.

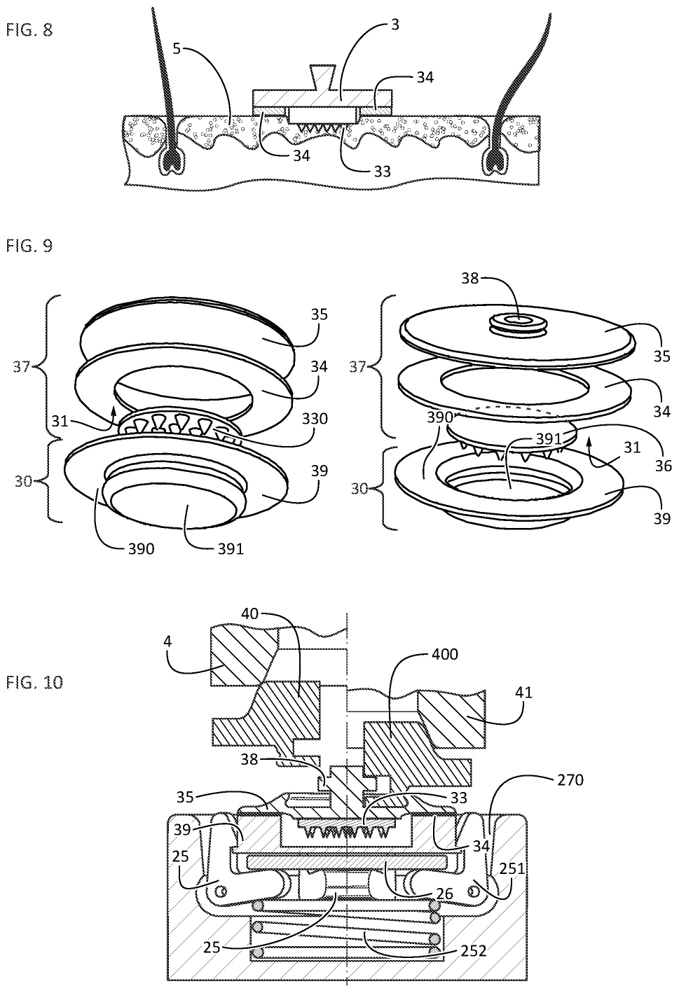

Referring to FIGS. 7 and 8, after the microneedle patch 3 has been mounted, that is an applicator 4 is provided with a microneedle patch 3 in a ready-for-application state, the microneedle patch 3 may be applied to the skin 5 of the subject, e.g. by the subject itself. In such as case, for example, prior to applying the microneedle patch, the skin may have been treated with alcohol and/or hair removed, such as by shaving, in the area where the microneedle patch is to be applied. The method may be performed by the subject to self-apply by the subject the microneedle patch 3 to its skin 5, without intervention of a medical practitioner, and can be part of a non-therapeutic, non-diagnostic and non-surgical procedure.

To apply the microneedle patch 3, the applicator 4 may be brought in a position suitable for applying the microneedle patch 3 on a selected part of the skin 5 of the subject, as is illustrated in FIG. 7. The applicator may e.g. be placed on a part of the body of the subject selected from the group: head, ear, neck, limb, arm, upper arm, lower arm, hand, leg, upper leg, lower leg, foot, torso, chest, abdomen, pelvic region, back, shoulders, buttocks. For example, the applicator may be placed on the inside of the lower arm. Thereby, a relatively low amount of force is needed to penetrate the skin, since the skin is relatively thin in that area, and additionally few preparations are required because this body part generally does not have that much hair, though a small shaving kit may still be provided with the system to remove any hair if necessary. The applicator may be selected for the thickness of the skin or fat percentage of the selected body part, and e.g. to exert more force on the microneedle patch if the applicator is for a body part with relatively thick skin layers, such as at a buttock, compared to the force of an applicator for a part with relatively thin skin layers, such as an ear. It will be apparent that, e.g. in case of self-administration, the body part is preferably within reach of the hands 6 of the subject.

With the applicator 4 in the selected position, the microneedle patch 3 may be applied to the skin 5. To that end a movement of the microneedle patch 3 relative to the skin 5 may be actuated. In FIG. 7, the movement is actuated by releasing a biasing spring 430 which has been pre-tensioned during the mounting of the microneedle patch on the applicator 4. The biasing spring 430 causes the microneedle patch 3 to move, in this example the releasing of a biasing spring 430 causes the coupling interface to move downwards relative to the base 47 of the applicator, which pushes the microneedle patch (which is still coupled to the coupling interface 40) downwards, as is illustrated with the arrow in FIG. 7. The movement of the microneedle patch 3 causes penetration into at least into the stratum corneum of the epidermis of the skin 5 with the microneedle(s) 33. The microneedle(s) 33 may penetrate the stratum corneum, and optionally, further into the epidermis until into one of the group selected from: the stratum lucidum, the stratum granulosum, the stratum spinonsum, the stratum basale, basement membrane. The microneedle(s) 33 may e.g. penetrate through the epidermis further into the skin 5, until into the dermis or into the hypodermis subcutis. The microneedles can e.g. penetrate the dermis until into the papillary dermis or into the reticular dermis.

As e.g. shown in FIG. 8, the applicator 4 brings the microneedle patch 3 onto the skin 5 with the skin-adhesive surface 34 contacting the skin. Upon this contact, the microneedle patch is tacked onto the skin 5 by the stiction of the adhesive surface. Shortly before, during or after the skin-adhesive surface 34 contacts the skin, the microneedle(s) 33 penetrate(s) the skin. The microneedle patch applied on the skin may then be used to exchange substances between the microneedle patch and the body of the subject through the area of the skin perforated by the microneedle(s). Through the microneedles 33 a pharmaceutically active substance to the subject may be administered. Alternatively, or additionally, the microneedle patch 3 may collect through the microneedles 33 a substance from the subject. For example, the method may comprise transdermal collecting of the substance from the subject, or transdermal administration of the ingredient to the subject. To that end, for example, the microneedle patch 3 may be provided with a reservoir for a pharmaceutically active ingredient, or to store and/or with a reservoir for a substance collected from the subject via he microneedle. Also, the microneedle patch 3 may be provided with a sensor for collecting data about the subject.

The microneedle patch 3 may generally be of any type suitable to perform operations between the microneedles 33 and the body of the subject, e.g. administer a pharmaceutically active substance to the subject, or collect, through the microneedles, a substance from the subject, e.g. dermally or transdermally, sense properties of the body or modify the body (e.g. by heating of, or sending electrical current into, the perforated area of the skin). The microneedle patch may comprise a reservoir for storing the substance to be delivered to or collected from the subject. The reservoir may e.g. store a pharmaceutically active ingredient, and the reservoir can for instance be connected to the microneedles for controlled transdermal release of the pharmaceutically active ingredient into the subject.

To perform the operations, microneedle patch 3 can be placed on the skin of a subject with the skin-adhesive surface contacting the skin, e.g. with an applicator as described above. The skin may have been stretched before placing microneedle patch or be stretched during placement. The microneedles 33 of the microneedle patch may upon, or after, contacting the skin penetrate at least the epidermis of the skin. For example, the microneedles may perforate the stratum corneum without piercing through, or pierce through the stratum corneum, and any intermediate layers, until into one of the following skin layers (without piercing that layer): stratum lucidum, stratum granulosum, stratum spinonsum, stratum basale, basement membrane, papillary dermis, reticular dermis.

The microneedle patch 3 may be of a type to be applied by a medical practitioner or be used to self-administer by a subject. In this respect, the subject can be a human or an animal. The microneedle patch may e.g. be applied on a part of the body of the subject selected from the group: head, ear, neck, limb, arm, upper arm, lower arm, hand, leg, upper leg, lower leg, foot, torso, chest, abdomen, pelvic region, back, shoulders, buttocks. For example, the microneedle patch 3 may be applied to the inside of the lower arm. Thereby, a relatively low amount of force is needed to penetrate the skin, since the skin is relatively thin in that area, and additionally few preparations are required because this body part has not that much hair. The applicator 4 may be adapted to the thickness of the skin of the selected body part, and e.g. to exert more force on the microneedle patch if the applicator is for a part with relatively thick skin layers, such as at a buttock, compared to the force of an applicator for a part with relatively thin skin layers, such as an ear. In case of self-administration, the body part is preferably within reach of the hands of the subject.

When the microneedle patch 3 is applied, the microneedles 33 perforate the skin, allowing substances to be exchanged between microneedle patch 3 and the skin. For example, pharmaceutically active ingredients or other substances may be administered through the skin barrier into the body or substances from the body collected into microneedle patch through the skin barrier. The administered substances can e.g. dissolve into the surrounding skin tissue and diffuse to the microcirculation of the skin for example or penetrate deeper into the body.

Generally speaking, the microneedle patch 3 may have any suitable type and number of microneedles. In FIG. 9, the microneedle patch 3 comprises for example an array 330 of microneedles 33 but less or more microneedles may be present, and the microneedles 33 may be provided in another arrangement.

The microneedles 33 may e.g. be solid, coated, hollow, bio-degradable or non-bio-degradable, or a mixture thereof, and have any other characteristics suitable for the specific implementation. The microneedles may be non-degradable needles with the pharmaceutically active ingredient embedded therein, such as porous needles which release an ingredient from the pores or, of needles where the ingredient diffuses out of the needle into the skin tissue, just to give some examples. In FIG. 9, the microneedles are part of a microneedle platform. The microneedle platform comprises a microneedle array, in which in this example a plurality of microneedles is arranged in rows and columns. This array allows to perforate a large area of the skin. The needles 33 project at the microneedle side 31 beyond the adhesive layer downwards. As shown, the platform comprises a rigid plate 36 provided with the microneedles 33 and from which the microneedles project. The microneedles 33 may be made of any material suitable for the application, such as silicon, metal, polymer, glass and ceramic, and in a variety of shapes and sizes. The microneedles may be manufactured using any technique suitable for the specific material, shape and size. For example, microfabrication techniques of adding, removing, and copying microstructures utilizing photolithographic processes, silicon etching, laser cutting, metal electroplating, metal electropolishing and micro-moulding may be used.

The microneedles 33 may have any suitable length and diameter. For example, a diameter of several tens to several hundreds of micrometres and/or a length of several tens, a few hundreds to a few thousands of micrometres have found to be suitable. Since the microneedle is relatively small in diameter and length as compared to the conventional needles, the activation of the nociceptors in the skin is reduced and preferably avoided completely. Thus, significant alleviation of pain experience by the subject can be obtained when dermally or transdermally administering drugs or taking samples from the subject. The physical damage to the skin is minimal as a consequence of the small dimensions of the needles. Preferably, the dimensions are such that, under non-occlusive conditions, the perforations close once the microneedles are removed within less than 2 days, preferably less than 1 day, such as in less than 10 hours, for example in several hours. The perforations may be micropores, e.g. of a diameter less than 500 micrometre.

The microneedle patch 3 may comprise, as illustrated in FIG. 9 a microneedle carrier 37 and a removable part 30 which are separable from each other to bring the patch 3 from the pre-application state into the ready-for-application state. The shown carrier 37 has a microneedle side 31, and the microneedle patch 3 has one, two or more microneedles 33 which project from the microneedle side when applied to the skin 3.

In this example, the carrier 37 is provided at the microneedle side 31 with a skin adhesive surface 34. In the pre-application state, the skin-adhesive surface 34 is covered, whereas in the ready-for-application state the surface 34 is exposed. The surface is coated in this example with a dermatologically acceptable type of adhesive, suitable to attach the microneedle patch 3 onto the skin 5 by the stiction of the skin-adhesive surface 34. The microneedle(s) 33 projects relative to the skin adhesive surface 34 out of the microneedle patch 3 at the microneedle side 31.

At the side opposite to the microneedle side 31, the carrier 37 is provided with a coupling interface 38 compatible with the coupling interface 40 of the applicator 4. In this example the coupling interface is shaped as a knob provided which the moving parts of the coupling interface 40 can grab and hold.

As shown, in this example the carrier 37 is disk-shaped but other shapes, such as rectangular or polygonal are also possible. The carrier 37 is in this example a multi-layer carrier, which comprises as a top layer a disk-shaped backing 35, which is separated from the microneedle side by a microneedle plate 36. In this example, the microneedle plate 36 is circumferentially enclosed by a flat annulus, which is provided with the skin-adhesive surface 34. The annulus may be flush with the microneedle plate 36 and the microneedle(s) project from the microneedle plate 36.

The removable part 30 comprises a cover 39 which in the pre-application state covers at least a part of the microneedle side 31, and which may be separated from the microneedle patch 3 after conditions (i) and (ii) have been met by the applicator 4, e.g. when the application removes the microneedle patch 3 from the docking platform 20. The cover 39 may be non-destructively releasably attached to the carrier 37 and engages when docked in the pre-application state with the latch 25 of the docking platform 20. The cover 39 may be arranged to be separated from the carrier 37 when a predetermined force may be exerted on the carrier 37 by the applicator 4 while the cover 39 may be held onto the docking platform 20, as explained above.

In this example, the cover 39 covers at least the tacking parts of the skin-adhesive surface 34. The exposed surface of the cover facing away from the skin-adhesive surface 34 is non-adhesive. Thus, the cover 39 prevents the patch 34 from sticking unintendedly prior to application. The part 39 covers the microneedles 33 of microneedle patch as well. The cover 39 thus allows to ensure that the microneedles are not contaminated with hazardous substances or micro-organisms prior to use. Shortly before applying microneedle patch 3 to the skin, the microneedle patch 3 may be brought into the ready for application state, in this example the cover 39 may then be removed to expose the adhesive surface 200, and the microneedles 330, by fulfilling conditions (iii) and (iv).

The cover 39 may generally be shaped in any manner suitable for the specific type of patch. In this example, the cover 39 covers the microneedle(s) 33 and is provided with a depression 391 in which the microneedle 33 can be admitted. The cover 39 has a contact area 390 outside the depression 391 which contacts the microneedle side 31, in this example which abuts in the pre-application state to the annulus 34. In the pre-application state, the microneedles project out of the microneedle side 31 beyond contact area 390 into the depression 391.

Referring now to FIG. 10, the example therein is the same as of FIGS. 1-6, but differs as follows. As shown, the table 26 lies recessed in the depression 27 and is movable in the vertical direction relative to the base 20. The levers 251 are rotatable mounted, rotatable around a horizontal axis and provided with a beam which projects in the horizontal direction. In the depression table 26 is supported in the depression 27 by the beam, as well as by a biasing spring 23. When the patch 3 is docked, the table is pushed into the depression, against the biasing force of spring 252, downwards in FIG. 10, which causes the levers to rotate, causing the projections 250 to lock the flange of the cover into the depression 27 and thereby docking the patch 3.

As illustrated in the left hand side of FIG. 10, the applicator 4 may then be brought into alignment with the patch, with the coupling interface 40 facing the matching part 38 of the patch 3. The applicator 4 may then be brought closer to the docking station 2, as illustrated at the right-hand side, in this example by lowering the applicator 4. This causes the coupling block 400 to be pushed in the opposite direction, inwards into the applicator 4, which due to the inclined, tapered entry causes the block 400 to move in the perpendicular direction as well, causing the knob to be clamped into the blocks 400. In this respect, the upwards projecting peripheral circumferential rim of the backing 35 is resiliently deformable. Thus, between the situation depicted at the left-hand side and the situation depicted at the right-hand side, this edge will resiliently deform and return into its initial shape, causing the coupling block 400 to “plop” into the annular space between the rim and the knob.

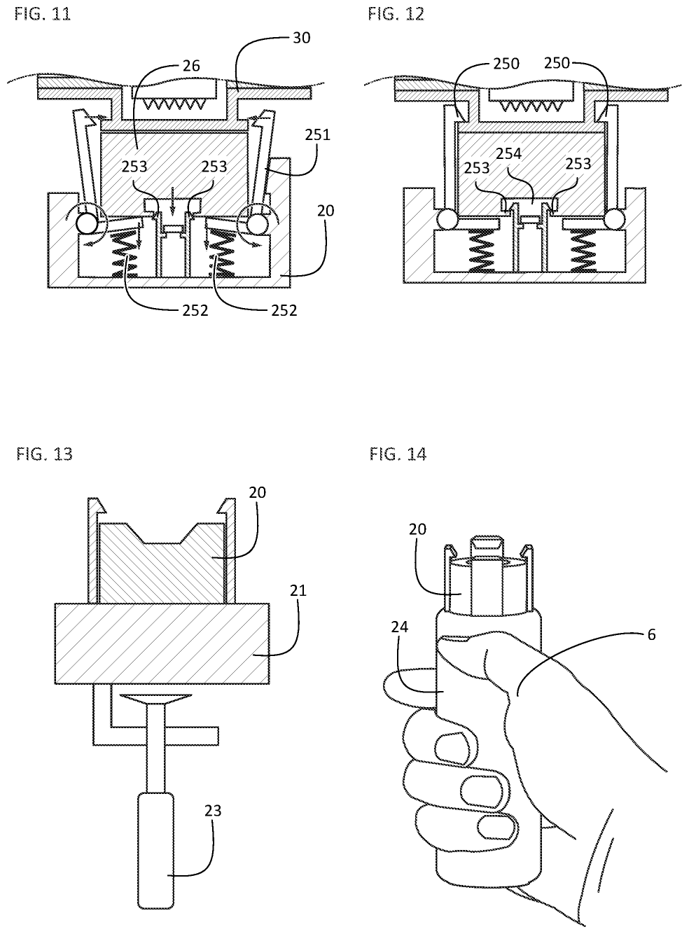

Referring now to FIGS. 11 and 12, the example therein is the same as of FIGS. 10, but differ as follows. The docking platform 20 has a bias stop 253 which holds the table 26 in the latching position. As shown, the table 26 is provided at the bottom, that is the side facing the depression with a recess 254 in which flexing hooks attached to the wall of the depression lock, to hold the table 26 against the biasing force of the spring 252. The interlocking of the hooks with the recess 254 causes an audible or haptic feedback which allows the user to perceive that the patch 3 is docked.

Referring now to FIGS. 13 and 14, the examples therein are the same as of FIGS. 1-7, but differ as follows. In the example of FIGS. 2-7, the base 21 has a weight exceeding the separation force. The separation force is the minimum force in the direction of gravity required to move the applicator 4 out of the predetermined position and take the microneedle patch 3 from the docking platform 20. However, as illustrated in FIG. 13, alternatively or additionally, for example, the docking station 2 further may comprise a fixation 23 for fixating the base 21 to a support, such as a table or a workbench.

The docking station 2 may also be without the base 21, and for example comprise a grip 24 for holding the docking station 2 manually in position while moving the applicator 4 out of the predetermined position, as is illustrated in FIG. 14. For example, the subject may hold the docking station in his hand 6, dock microneedle patch and then slide with the other hand the applicator 4 on the docking platform 20, for instance.

For instance, using the example of FIG. 14, the docking station 2 can be turned upside down to dock in the docking platform 20 a microneedle 3 which lays in or on a support, such as a tray or a box, and which is in the pre-application state. The docking station 2 can e.g. be turned such that the docking platform 20 faces the microneedle patch 3, and the docking platform 20 be brought into physical contact with the microneedle patch 3 to mount the microneedle patch 3 on the docking platform. Once the microneedle patch 3 is docked, the docking station 2 can be turned upside up, such that the microneedle patch 3 docked in the docking platform is on top. For example, this can be used to take a selected microneedle patch 3 out of a tray with an array of patches.

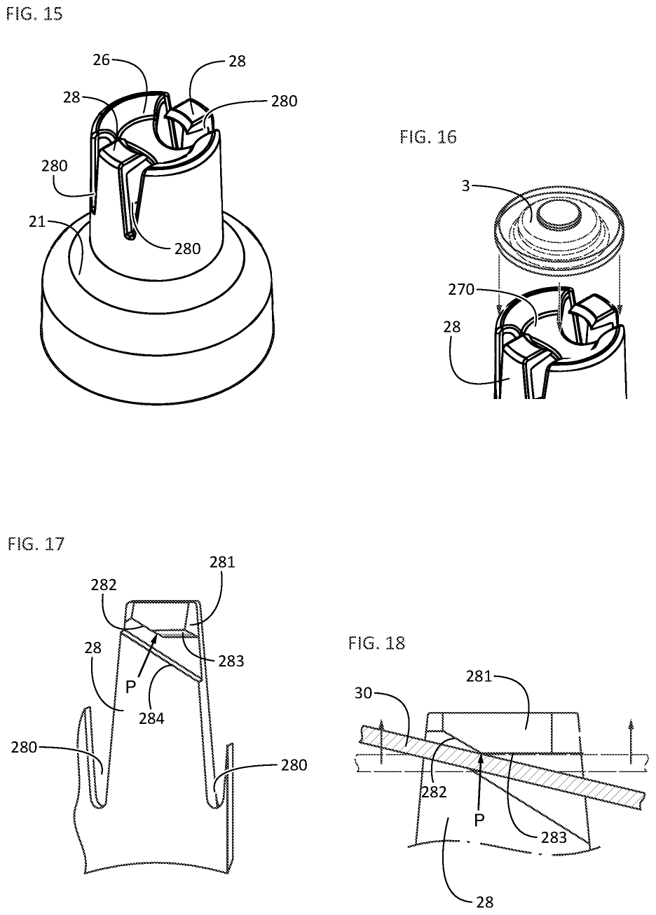

Referring now to FIG. 15-17, the example therein is the same as of FIGS. 1-7, but differ as follows. As shown, the table 26 comprises a bridge between opposite sides of the top 22 of the base 21, with depression 270 in the middle of the bridge. At both longitudinal sides of the bridge, a respective fastener 28 is present. The fasteners 28 are separated from the table by respective vertical slots 280 in the base 21 which extend from the top 22 up to the foot of the fastener 28, each fastener 28 separating two of such slots. As illustrated in FIG. 16, the patch 3 can be docked by placing the patch on the top 22, and pushing the patch 3 onto the bridge such that the fasteners 28 flex outwards and then back inwards to latch the patch 3. As in the example of FIGS. 1-7, the applicator 4 may be placed over the docking station, and once the sheath 41 is moved over the docking station the diameter of the sheath 41 restricts outwards movement of the fasteners 28 which inhibits unlatching of the latched patch 3.

As illustrated in FIGS. 17 and 18, the fasteners 28 may have at the top side 22 a projection 281 which projects inwards, i.e. perpendicular to the longitudinal direction of the fastener and towards the bridge. The bottom side of the projection 281 projects over the rim of the removable part. The bottom side has an inclined plane 282 and a parallel plane 283, the parallel plane being more or less parallel to the longitudinal direction of the bridge that forms table 26. When the patch 3 is coupled to the applicator 4 and moved upwards, this bottom side contacts the rim, as illustrated in FIG. 18. As shown in FIG. 18, the force exerted on the rim by the bottom side will cause the removable part to pivot around point P, and thereby be peeled off the patch 3, or when the cover does not deform, causes the removable part at least to be unevenly loaded and to dislodge the cover from the adhesive. This reduces the amount of force required to separate the removable part from the patch 3. As shown, the fastener 28 has a step 284 a bit lower than the planes 282,283 which prevents the pivoting beyond a predetermined angle.

The system 1 may be provided e.g. with or without the microneedle patch 3. For example, the microneedle patch 3 may be present in the package in which the system 1 may be provided. Alternatively, or additionally, the microneedle patch 3 may be provided separately. The system 1 may comprise a set of microneedle patches in the pre-application state.

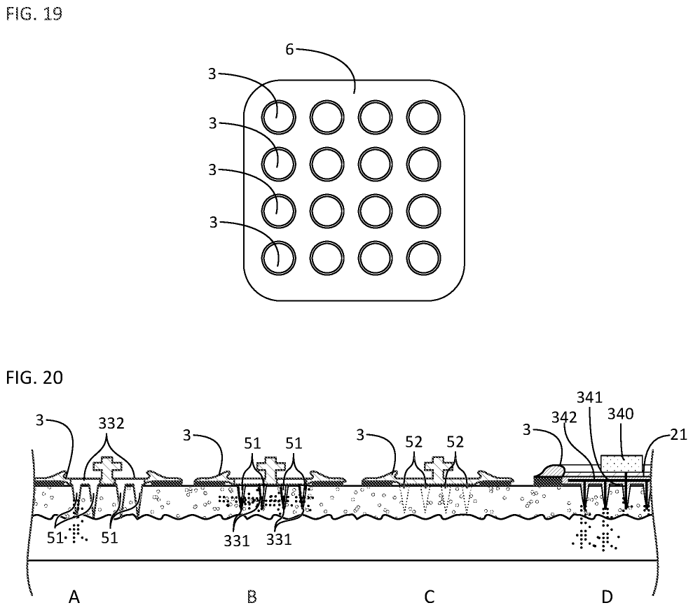

The microneedle of the microneedle patch 3 may be sterile in the pre-application state. The system 1 may comprise a sterile package in which the microneedle patch 3 is provided, with at least the microneedle 33 or array 330 being sterile. The package may be provided with instructions to perform a method of applying the microneedle patch 3 with the system 1, such as described above. FIG. 19 show for example a tray in which the microneedles are located in such a sterile condition. FIG. 19 shows as an example of a package for microneedle patches, a tray 6 with recesses. In each recess a patch 3 is present, in this example with the removable part 30 oriented upwards to be able to engage with the docking station and be docked therein without requiring manipulation of microneedle patch 3 relative to the tray 6. To take a respective patch, the docking station may be placed on the tray, with the docking platform over the recess. The docking platform may then be brought to contact the cover 39 to e.g. clamp or otherwise attach to microneedle patch and be moved away from the tray, taking microneedle patch out of the recess.

FIG. 20 shows several examples of implementations of the microneedle patch. In the examples A and B of FIG. 20, the applied patch 3 has removable microneedles and after removing the needles has an open region 332 through which the skin 5 is exposed. More specifically, although the open region 332 may be larger or smaller than this area, this open region 332 corresponds to the area of the skin where the exposed skin 5 has been penetrated with microneedles. After penetration, the microneedles have been removed, leaving the skin 5 with corresponding holes or perforations 51 and the perforated area exposed. Although other types of microneedles could be removed as well, in the example A, the microneedles are solid needles which simply perforate the skin 5. As shown, after perforation of the skin the microneedles are removed but the perforations 51 remain for a certain period of time. During this period, e.g. substances can be administered by applying a gel or cream on the skin 5 in the perforated area.

In example A, the microneedles are removed from the applied patch 3 while the remaining parts of microneedle patch 3 are kept in place on the skin 5. These parts inhibit relaxation of the skin 5 in the shielded, open region 332. Thus, undesired closure of the perforations 51 due to the relaxation can be reduced, and the conditions of applying substances can be more controlled.

In the example B of FIG. 20, the microneedles have been provided with a coating 331 with a pharmaceutically active ingredient which is released into the skin 5 after perforation thereof. Depending on the specific implementation, such a coating 331 may adhere to the walls of the holes 51 created in the skin 5, and thus be transferred from microneedle patch into the skin tissue. This allows a continued release while the skin heals, or alternatively the microneedles may be kept in place until the coating has released the desired amount of pharmaceutically active ingredient. The remaining parts of microneedle patch 3 are kept in place on the skin 5 and inhibit relaxation of the skin, which prevents undesired movement of the substances released from the coating and/or the transferred coating, e.g. due to repetitive stretching and relaxation of the skin 5 under microneedle patch 3 when the subject moves body parts.

In the example C, microneedle patch 3 is provided with microneedles made of a bio-degradable material, for instance soluble into the skin tissue, with a pharmaceutically active ingredient embedded therein. The ingredient may e.g. by embedded in a matrix but likewise inside the microneedle a separate reservoir filled with a formulation containing the pharmaceutically active ingredient may have been provided. The microneedles are not actively removed but bio-degrade. When the microneedles dissolve into the skin tissue or otherwise bio-degrade after application of microneedle patch 3, the pharmaceutically active ingredient embedded in the microneedles is released in the regions 52.

In the example D of FIG. 20, the microneedles of the microneedle patch 3 remain in the skin during the treatment and are not removed from microneedle patch 3. The microneedle patch 3 is provided with a fluid transport system 340 which comprises a reservoir 341 e.g. for a substance to be administered to or collected from the subject. The microneedles are implemented as a base with one or more projecting needles inside which channels 342 are present. Through the channels 342 a substance can be administered from the reservoir 341. Alternatively, the reservoir 341 may initially be empty and be used to store a substance collected from the subject. For example, a substance may be collected transdermally from the subject, e.g. by applying microneedle patch during a predetermined period of time and subsequently removing microneedle patch 3 from the skin 5, where during the period a bodily fluid flows through the channels 342 into the reservoir 341. It will be apparent that in the example D, instead of the reservoir 341, e.g. swellable microneedles may be used which swell with interstitial fluid when applied into the skin and may be taken from the skin 5 for e.g. further analysis.

In the foregoing specification, the invention has been described with reference to specific examples of embodiments of the invention. It will, however, be evident that these are merely illustrative to elucidate the invention, and that various modifications and changes can be made without departing from the broader scope of the invention as set forth in the appended claims.

For example, the system can further comprise a package in which the applicator and/or the docking platform are provided, and e.g. of a suitable packaging material such as plastic. The package may be a sterile package and alternatively or additionally be provided with instructions as to how to dock microneedle patch on the docking system and/or how to mount the docked patch on the applicator and/or how use and place the applicator on a skin, as well as with instructions of treatment of a condition such as dosing and frequency of application for a specific condition. The package can e.g. be a sterile sealed bag of a suitable material, such as plastic. In such a case, the microneedle patches may be separately, and optionally individually, packaged.

Also, the microneedles may pierce completely through a layer or penetrate into the layer without piercing through the layer. The microneedles may for example penetrate deeper into the skin, and pierce through the epidermis, until into the dermis or into the hypodermis subcutis. The microneedles can for example penetrate the dermis until into the papillary dermis or until into the reticular dermis. The microneedles can e.g. pierce the stratum corneum, and any intermediate layers, until into one of the following skin layers without piercing that layer: stratum lucidum, stratum granulosum, stratum spinonsum, stratum basale, basement membrane, papillary dermis, reticular dermis. Preferably, but not necessarily, the penetration of the microneedle(s) does not activate nociceptors in the skin, or at least not above the threshold the subject perceives a sensation of pain.

Moreover, the terms “front,” “back,” “top,” “bottom,” “over,” “under” and the like in the description and in the claims, if any, are used for descriptive purposes and not necessarily for describing permanent relative positions. It is understood that the terms so used are interchangeable under appropriate circumstances such that the embodiments of the invention described herein are, for example, capable of operation in other orientations than those illustrated or otherwise described herein.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements The mere fact that certain measures are recited in mutually different claims does not indicate that a combination of these measures cannot be used to advantage.

### LIST OF REFERENCE NUMBERS

1 microneedle patch applicator system
2 docking station
3 microneedle patch
4 applicator
5 skin
6 patch tray
20 docking platform
21 base
22 top side
23 fixation
24 grip
25 latch
26 table
27 depression
28 fastener
29 applicator guide
30 removable part
31 microneedle side
32 applicator coupling side
33 microneedle
34 adhesive surface
35 backing
36 microneedle plate
37 microneedle carrier
38 coupling interface
39 cover
40 coupling interface
41 sheath
42 actuator
43 energy storage
44 transducer
45 housing
46 movable platform
47 base
51 perforations
52 regions
250 projection
251 lever
252 biasing spring
253 bias stop
254 space
280 slot
281 projection
282 inclined plane
283 parallel plane
284 groove
300 projecting rim
301 exposed top
330 microneedle array
331 coating
332 open region
340 fluid transport system
341 reservoir
342 channel
390 contact area
400 coupling block
401 separating spring
430 spring
431 spring latch
470 skin-side end
471 microneedle patch holder
472 actuator interface

1. A microneedle patch applicator system, comprising:
an applicator for applying, by a user of the system, a microneedle patch to a skin of a subject, the applicator comprising:
a coupling interface for coupling to the microneedle patch when the applicator is brought in a, predetermined, coupling position relative to the microneedle patch, and when the microneedle patch in a pre-application state in which the microneedle patch is not ready-for-application to a skin of a subject; and
wherein the applicator is arranged to transfer the microneedle patch from the pre-application state into a state ready-for-application, such that the microneedle patch is in said ready-for-application state when the microneedle patch is coupled to the applicator and the applicator has been moved out of the coupling position.

2. The system of claim 1,
comprising a docking platform for docking the microneedle patch in the pre-application state; and
wherein the coupling interface is arranged to couple to the microneedle patch when all of the following conditions (i)-(ii) are met:
(i) the microneedle patch is docked on the docking platform,
(ii) the applicator is brought in a predetermined position relative to the docking platform; and
wherein the docking platform and the applicator are arranged to collaborate to transfer the microneedle patch from the pre-application state into the state ready-for-application, such that the microneedle patch is in the ready-for-application state when all of the following conditions (iii)-(iv) are met:
(iii) the microneedle patch is coupled to the applicator;
(iv) the applicator has been moved out of the predetermined position and the microneedle patch has thereby been taken from the docking platform.

3. The system of claim 2, wherein:
the docking platform and the applicator are movable relative to each other by manual force of the user,
the docking platform and the applicator are arranged to transfer the microneedle patch from the pre-application state into the ready-for application when a predetermined manual manipulation of the docking platform and/or the applicator is performed by the user.

4. The system of claim 2, comprising a docking station, the docking system comprising:
the docking platform, and
a base with a top side on which the docking platform is located; and
wherein the docking platform is shaped to dock the microneedle patch:
oriented with a microneedle side of the microneedle patch facing towards the base, the microneedle side provided with a microneedle, and
oriented with an applicator coupling side of the microneedle patch facing away from the base, the applicator coupling side further facing away from the microneedle side.

5-7. (canceled)

8. The system of claim 2, wherein the docking platform is arranged to:
latch a microneedle patch which comprises in the pre-application state a removable part, which has to be removed to bring the microneedle patch into the ready-for-application state, and
retain the removable part on the docking platform when the applicator is being moved out of the predetermined position and the microneedle patch is being taken from the docking platform.

9. (canceled)

10. The system of claim 8, wherein the docking platform comprises a latch which latches upon docking the microneedle patch onto the table, and which is locked upon placing the applicator into the predetermined position; and, wherein:
the applicator comprises a sheath for sliding over the top-side of the docking platform, and
the latch comprises a resiliently or plastically deformable fastener which prior to sliding is movable in an inwards direction perpendicular to a sliding direction of the sheath, to latch the microneedle patch and the sheath when slid over the top-side blocking an outwards movement opposite to the inwards movement thereby locking the latch.

11-12. (canceled)

13. The system of claim 10, wherein the applicator comprises an actuator for actuating, when in operation, a movement of the microneedle patch in the ready-for-application state relative to the skin to penetrate at least into, or through, the stratum corneum of the epidermis of the skin with the microneedle; and wherein the applicator comprises an energy storage for storing energy to drive the actuator, and the energy is stored upon bringing the applicator in the predetermined position.

14. (canceled)

15. The system of claim 13, wherein the applicator comprises a transducer for converting kinetic energy of the applicator into potential energy stored in the energy storage; and wherein the applicator comprises:
a housing, and
a movable platform for holding the microneedle patch when coupled to the applicator, the movable platform being moved relative to the housing when the applicator is brought into the predetermined position, and
wherein the energy storage comprises a spring which is resiliently deformed by the movement of the movable platform to store the potential energy by bringing the applicator into the predetermined position,
a spring latch for latching the spring in tensioned state when the microneedle patch is transferred to the state ready-for-application and for releasing the spring to actuate the movement of the movable platform upon a point in time selected by the user, to place the microneedle onto the skin and penetrate the skin with the microneedles.

16. (canceled)

17. The system of claim 1, wherein the applicator comprises:
a base for positioning onto the skin, the base comprising:
a skin-side end;
a holder for holding the microneedle patch in the ready-for application state in position relative to the skin-side end, and
an interface for an actuator, for actuating a movement of the microneedle patch relative to the skin-side end to penetrate at least into the stratum corneum of the epidermis of the skin with the microneedle.

18. The system of claim 1, wherein:
in the pre-application state the microneedle patch comprises a microneedle carrier provided with a microneedle array at a microneedle side of the carrier, and a cover covering at least a part of the microneedle side;
the applicator further arranged to separate the cover from the microneedle patch.

19. The system of claim 1, comprising a guide for the applicator, the guide defining a predetermined path for moving the application from an initial position into the predetermined position.

20. (canceled)

21. The system of claim 1, wherein the applicator is arranged to transfer the microneedle patch into the ready-for application state without human contact to the microneedle patch.

22. (canceled)

23. The system of claim 1, comprising a set of microneedle patches in the pre-application state.

24-30. (canceled)

31. A microneedle patch for a system as claimed in claim 1; comprising a microneedle carrier with a microneedle provided at a microneedle side of the microneedle carrier, and a removable cover covering at least a part of the microneedle side; wherein the carrier is provided at the microneedle side with an adhesive surface which in pre-application state is covered and in the ready-for-application state is exposed, for attaching the microneedle patch onto the skin by the stiction of the adhesive surface.

32-34. (canceled)

35. The microneedle patch of claim 31, wherein at least one microneedle projects relative to the skin-adhesive surface out of the microneedle patch at the microneedle side.

36. The microneedle patch of claim 31, wherein the cover is non-destructively releasably attached to the carrier and engages when docked in the pre-application state with a latch of the docking platform.

37. The microneedle patch of claim 31, wherein the cover is arranged to be separated from the carrier when a predetermined force is exerted on the carrier by the applicator while the cover is held onto the docking platform.

38-40. (canceled)

41. The microneedle patch of claim 31, wherein the cover covers the microneedle and the cover has a depression in which the microneedle is admitted, and a contact area outside the depression which contacts the microneedle side and the microneedles project out of the microneedle side beyond contact area.

42-45. (canceled)

46. A method of mounting a microneedle patch on an applicator, comprising:
when the microneedle patch is in a pre-application state, in which pre-application state the microneedle patch is not ready-for-application to a skin of a subject, bringing an applicator for applying the microneedle patch to the skin of the subject in a coupling position relative to the microneedle patch, thereby coupling a coupling interface of the applicator to the microneedle patch; and
the applicator transferring the microneedle patch from the pre-application state into a ready-for-application state upon bringing the applicator into the coupling position or upon moving the applicator out of the coupling position.

47. The method of claim 46, wherein a system as claimed in claim 2, is used and the method comprises:
wherein the coupling interface is coupled to the microneedle patch when all of the following conditions (i)-(ii) have been met:
(i) the microneedle patch is docked on the docking platform,
(ii) the applicator is brought in a predetermined position relative to the docking platform; and
wherein the docking platform and the applicator collaborate to transfer the microneedle patch from the pre-application state into the state ready-for-application, such that the microneedle patch is in the ready-for-application state when all of the following conditions (iii)-(iv) are met:
(iii) the microneedle patch is coupled to the applicator,
(iv) the applicator has been moved out of the predetermined position and the microneedle patch has been taken from the docking platform.

48-60. (canceled)
